(19) United States
(12) Patent Application Publication
(10) Pub. No.: US 2023/0298175 A1
(43) Pub. Date: Sep. 21, 2023

(54) MACHINE LEARNING MODEL BASED METHOD AND ANALYSIS SYSTEM FOR PERFORMING COVID-19 TESTING ACCORDING TO EYE IMAGE CAPTURED BY SMARTPHONE

(71) Applicant: Fredrick James LEWIS, Pflugerville, TX (US)

(72) Inventors: Fredrick James LEWIS, Pflugerville, TX (US); Abhishek POTNIS, Mumbai (IN)

(21) Appl. No.: 18/321,722

(22) Filed: May 22, 2023

Related U.S. Application Data
(63) Continuation-in-part of application No. 17/531,744, filed on Nov. 20, 2021.
(60) Provisional application No. 63/116,816, filed on Nov. 21, 2020.
(60) Provisional application No. 63/344,536, filed on May 20, 2022.

(51) Int. Cl. G06T 7/00; A61B 3/113

(52) U.S. Cl. CPC G06T 7/0012; A61B 3/113; G06T 2207/30041; G06T 2207/20084

(57) ABSTRACT

A computer-implemented method and analysis system for performing a coronavirus test using a deep convolution neural network (DCNN) is provided. The method entails receiving examination data from a user's mobile computing device, which comprises the mobile computing device's identification information and an initial eye image captured by performing a fundus photography or an eye region photography via the mobile computing device's optical sensor; pre-processing the initial eye image to create an enhanced processed eye image; assessing the processed eye image by inputting it into a ML model that determines whether the eye image shows characteristics of being coronavirus positive; and returning the assessment result and the identification information to the original mobile computing device or another electronic device.

### CROSS-REFERENCE TO RELATED APPLICATIONS

The present application is a Continuation-in-part (CIP) application of U.S. patent application Ser. No. 17/531,744 filed Nov. 20, 2021, which claimed priority to U.S. Patent Application No. 63/116,816 filed Nov. 20, 2020; the disclosures of which are incorporated herein by reference in their entirety. The present application also claim priority to U.S. Patent Application No. 63/344,536 filed May 20, 2022; the disclosure of which is incorporated herein by reference in its entirety.

### FIELD OF THE INVENTION

The present invention generally relates to the field of coronavirus, including COVID-19, testing, and in particular to a method for a computer-implemented analysis system based on a machine learning (ML) model using a Deep Convolution Neural Network (DCNN), a Support Vector Machine (SVM), or a combination thereof. More specifically, the present invention relates to techniques of conducting ML model-based coronavirus infection probability assessments using eye images created from photographs of the eye captured by mobile computing devices such as smartphones.

### BACKGROUND OF THE INVENTION

Artificial intelligence (AI) has positively impacted countless domains, from assisting healthcare workers to detecting hostile military agents. Amid the ongoing COVID-19 pandemic, there is a dire need to develop diagnostic tests for coronavirus that are non-invasive and rapid yet still effective. Recent attempts to use AI for diagnosing coronavirus have trained neural networks to analyze chest X-rays. However, X-rays require resources only available in limited settings: dedicated space, an elaborate equipment set-up, and trained technicians. This presents an obstacle to implementing AI solutions at a scale large enough to match the pandemic's worldwide scope. Therefore, there is a need in the art for new coronavirus tests that use an easily accessible data source for AI analysis.

### SUMMARY OF THE INVENTION

The present disclosure provides novel eye/retinal imaging methods coupled with AI analysis to diagnose coronavirus infection. It outlines how smartphones can conveniently capture eye images by photographing the eye's fundus or the eye's region with a conventional handheld indirect ophthalmoscopy lens. The widespread availability of smartphones would encourage implementing and adopting the present invention, and the eye image capturing methods and AI analysis to diagnose coronavirus infection can be implemented with other mobile computing devices including, but not limited to, personal computers, laptop computers, tablet computers, electronic kiosks, and other specially configured computing devices having integrated or electrical connections to optical sensors and data processing circuitries necessary for the execution of the eye image capturing methods coupled with AI analysis to diagnose coronavirus infection in accordance with the embodiments of the present invention.

In each embodiment of the present invention, a computational method (coronavirus infection probability assessment method) for a coronavirus diagnostic test using a Machine Learning (ML) model comprising at least one of a Deep Convolution Neural Network (DCNN) and a Support Vector Machine (SVM) is provided. The method entails receiving examination data from a mobile computing device, which comprises the mobile computing device's identification information and an initial eye image captured by performing, without limitation, a fundus photography or an eye region photography via the mobile computing device's optical sensor; pre-processing the initial eye image to create an processed eye image; assessing the processed eye image by inputting it into the ML model that determines whether the processed eye image shows characteristics of coronavirus infection; and returning the assessment result and the identification information to the original mobile computing device and/or another electronic device.

In one embodiment, the pre-processing of the initial eye image comprises a pre-processing pipeline, which incorporates a novel and unique method of framing an eye image. In an image framing step, the eye region in the image is focused upon. A traditional eye image may have some parts of the nose, forehead, etc. visible in it, which are basically unimportant regions as they do not contain any distinguishing features for the detection of coronavirus infection and hence resulting in additional noise data. Traditionally, to deal with such scenarios, a framing pipeline is used to get the desired region. The present invention adds a novelty of framing the images by applying a color mask.

In one embodiment, the image framing step utilizes a YoloV5 model for CNN-based object detector. YoloV5 is a family of highly efficient object detection architectures and models. Transfer learning is used to train/tune the YoloV5 model on eye images from a training dataset so that it detects the eye region in an image. The trained/tuned YoloV5 model determines the coordinates of the eye region, if present, in an image. After getting the coordinates of the eye region, the portion outside of the detected coordinates is masked with a shade of light green color. By doing so, the aspect ratio, dimensions, and quality of the image are preserved. The eye region is not just cropped out because when doing so, the dimensions and aspect ratio changes and the quality of image degrades, which then affects the performance of the YoloV5 model. To mitigate this problem, and improve the quality of the framed image and maintain its dimensionality and aspect ratio, the portion outside the framed part is not removed but masked with a light green color.

Light green color is selected apart from other color such as black, white, brown, etc. for masking the non-eye region. It is because after careful analysis of the eye images in the training dataset, light green color is found to be not present in the original images in the training dataset. If another color such as brown, black, or white color is used for masking the non-eye region, there will be a possibility that the model would misinterpret it for some feature/area present inside the eye region having that color. Nonetheless, an ordinarily skilled person in the art shall appreciate that colors other than light green can be used for masking the non-eye region, as long as the chosen masking color is not one of possible colors present in human eyes.

Furthermore, an analysis system for performing a coronavirus test using a ML model comprising a DCNN, a SVM model, or a combination thereof is provided. The analysis system comprises a mobile computing device configured to capture an eye image by a fundus photography, which is not mandatory, or an eye region photography via an optical sensor of the mobile computing device; an electronic device; and an analysis server. The analysis server includes a communication circuit unit, a storage circuit unit and a processor. The communication circuit unit is configured to establish a network connection to the mobile computing device and the electronic device. The storage circuit unit is configured to store programs and a database. The processor is configured to access and execute the programs that perform a coronavirus infection probability assessment method. To ensure scalability and availability of the system, the training, testing, and run-time execution of the ML model may be implemented by Cloud services (e.g., Amazon® Web Services (AWS)). For example, in the training and testing of the ML model, AWS Sagemaker is used. A WebApp may also be deployed, which is an ec2 instance in AWS hosting the trained ML model, for executing the assessment of the new image.

### BRIEF DESCRIPTION OF THE DRAWINGS

Embodiments of the invention are described in more details hereinafter with reference to the drawings, in which:

FIG. 1 depicts a block diagram illustrating an analysis system for coronavirus infection probability assessment based on eye image captured by a mobile computing device in accordance with one embodiment of the present invention;

FIG. 2 depicts a flowchart of a coronavirus infection probability assessment method using a ML model on the eye image captured by a mobile computing device;

FIG. 3 depicts a schematic diagram illustrating the architecture of the DCNN model of the ML model;

FIG. 4 depicts a schematic diagram illustrating the architecture of the SVM model of the ML model;

FIG. 5 depicts a flowchart of a training method for the ML model;

FIG. 6 depicts a series of exemplary eye images illustrating an image pre-processing in accordance with one embodiment of the present invention;

FIG. 7 depicts a schematic diagram illustrating the architecture of a combined CNN and SVM model as the ML model; and

FIG. 8 depicts a schematic diagram illustrating a Master-Meta model using multiple ML model predictions.

### DETAILED DESCRIPTION

This description sets forth a method and system for coronavirus infection probability assessment using a ML model comprising a DCNN, a SVM, or a combination thereof and the likes as preferred examples. Those familiar with the art will understand that modifications, additions and/or substitutions may be made without departing from the scope and spirit of the invention. Specific details may be omitted so as not to obscure the invention; however, the disclosure is written to enable someone knowledgeable with the art to implement these concepts without excessive experimentation.

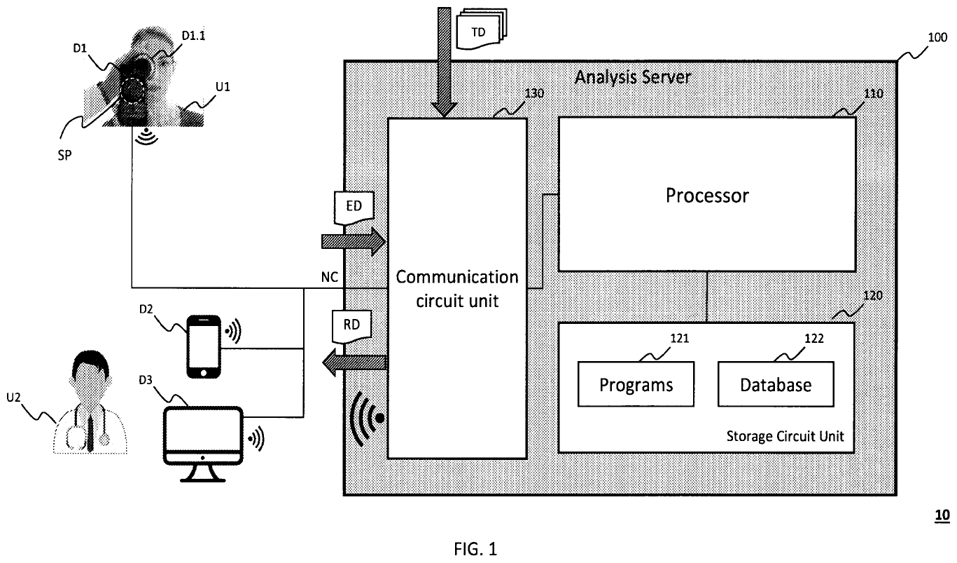

Referring to FIG. 1 for the following description. In one embodiment, analysis system 10 (also called as iDetect system) comprises a mobile computing device D1, an analysis server 100 and an electronic device D2. The analysis server includes a processor 110, a storage circuit unit 120 and a communication circuit unit 130. The mobile computing device D1 may capture an eye image (or scanned picture) SP on the eye of a user U1 via a camera that has a handheld indirect ophthalmoscopy lens D1.1 equipped. The mobile computing device D1 may send examination data ED with the captured eye image SP and identification information for the mobile computing device (or the user U1) to the analysis server 100 via the network connection NC (by a running client application or a transactional website corresponding to the analysis server).

The communication circuit unit 130 is configured to establish a network connection NC to the mobile computing device D1 and the electronic device D2 (e.g., a smartphone used by medical personnel U2). In another embodiment, the analysis system 10 could further include a terminal device D3 (e.g., a computer used by the medical personnel U2) having a network connection NC with the communication circuit unit 130. Additionally, the analysis server 100 may receive data (e.g., training data TD) via the internet through the communication circuit unit 130. It has a wireless module to enable Wi-Fi communication technology. The communication circuit unit 130 could further support other communication technologies, such as the Global System for Mobile Communication system or Bluetooth, and the invention is not limited hereto.

The storage circuit unit 120 is configured to store data, for example programs 121 and database 122. The storage circuit unit 120 may also store firmware to manage the analysis server 130. The database 122 may record training data TD, the trained or untrained ML model, examination data ED, and/or result data RD. In the embodiment, the storage circuit unit 120 may be any type of hard disk drive or non-volatile memory storage device (e.g., solid state drive). A dynamic random access memory (DRAM) for temporarily storing data may be integrated in the storage circuit unit 120 or be disposed in the analysis server 100.

In the embodiment, the processor 110 is hardware with computing capability, used to manage the analysis server's overall operation 100. The processor 110 may be, for example, a central processing unit (CPU) with one core or multiple cores, a microprocessor, or other programmable processing unit, such as a digital signal processor, programmable controller, application-specific integrated circuits, programmable logic device, or similar devices.

The processor 110 may access and execute the programs 121 to execute the coronavirus assessment method (or coronavirus testing method) provided by this disclosure, the steps of which are explained below with subsequent figures. In this regard, some supporting references for the correlation between retina patterns and coronavirus viral infection are provided as follows. Ivan Seah and Rupesh Agrawal, “Can the Coronavirus Disease 2019 (coronavirus) Affect the Eyes? A Review of Coronaviruses and Ocular Implications in Humans and Animals”, Ocular

Immunology and Inflammation, 28:3, 391-395, DOI:10.1080/09273948.2020.1738501. Yun Wang et al., “The Role of Apoptosis within the Retina of Coronavirus-Infected Mice”, Investigative Ophthalmology & Visual Science, September 2000, Vol. 41, No. 10. Tom Tooma, “Does Coronavirus Affect Your Eyes or Vision?”, https://www.nvisioncenters.com/coronavirus-and-the-eyes. All references above are incorporated herein by reference in their entities.

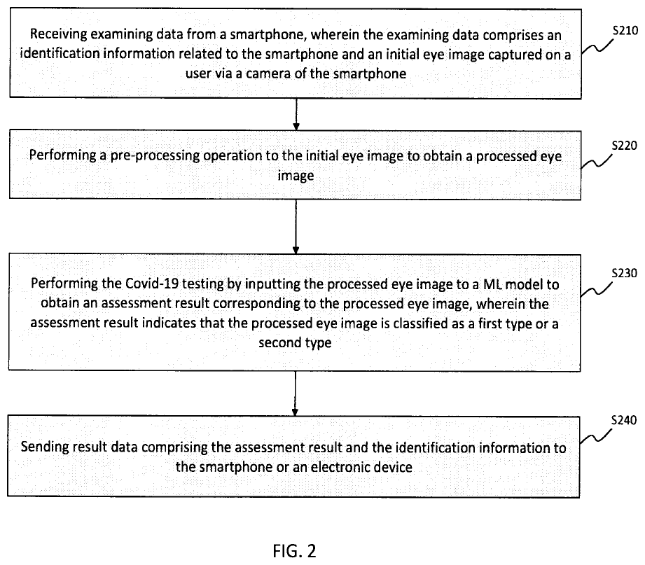

Referring to FIG. 2 for the following description. In step S210 the processor 110 receives examining data ED from a mobile computing device D1, which comprises the identification information of the mobile computing device D1 and an initial eye image captured on the user U1 with the s mobile computing device's optical sensor. In another embodiment, the identification information is related to the user U1.

The initial eye image comprises: an initial retinal image captured by performing a fundus photography on the user via the optical sensor of the mobile computing device; or an initial eye region image captured by performing an eye region photography via the optical sensor of the mobile computing device. The eye region includes at least the retina, fundus, sclera, pupil, iris, cornea, conjunctiva, and lens of an eye.

Next, in step S220, the processor 110 pre-processes the initial eye image to create an enhanced processed eye image. This pre-processing operation corrects any flaws in the initial eye image, such as being out-of-focus, underexposed, and/or overexposed. In another embodiment, the pre-processing step may further transform the initial eye image to conform to predefined specifications for a fixed size and image standard, which would allow more efficient processing for the ML model. For example, the processed eye image may be edited to 512×512 pixels consisting of three channels of RGB (red, green, blue) information. However, the invention is not limited hereto, for example, the resolution of the initial eye image or the processed eye image can be other suitable scales more than the 512×512. If the processor 110 determines that the initial eye image is out-of-focus, it can perform a super resolution operation on it to create the processed eye image. Or, if the processor 110 determines that the initial eye image is underexposed or overexposed, it can adjust the contrast and brightness values so the processed eye image will have values within a predefined range.

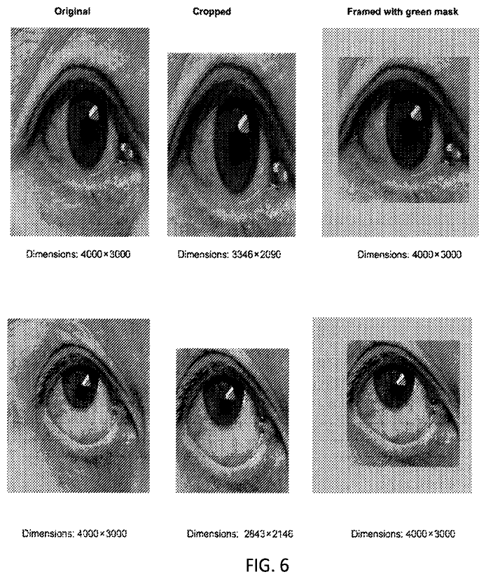

Referring FIG. 6 for the following description. In one embodiment, the pre-processing of the initial eye image comprises a pre-processing pipeline, which incorporates a novel and unique method of framing an eye image. In an image framing step, the eye region in the image is focused upon. A traditional eye image may have some parts of the nose, forehead, etc. visible in it, which are basically unimportant regions as they do not contain any distinguishing features for the detection of coronavirus infection and hence resulting in additional noise data. Traditionally, to deal with such scenarios, a framing pipeline is used to get the desired region. The present invention adds a novelty of framing the images by applying a color mask.

In one embodiment, the image framing step utilizes a YoloV5 model for CNN-based Object Detector. YoloV5 is a family of highly efficient object detection architectures and models pretrained on the COCO dataset. Transfer learning is used to train/tune the YoloV5 model on eye images from a training dataset so that it detects the eye region in an image. The trained/tuned YoloV5 model determines the coordinates of the eye region, if present, in an image. After getting the coordinates of the eye region, the portion outside the detected coordinates is masked with a light green shade. By doing so, the aspect ratio, dimensions, and quality of the image are preserved. The eye region is not cropped out because when the image is cropped, the dimensions and aspect ratio changes and the quality of image degrades, which then affects the performance of the YoloV5 model. To mitigate this problem, and improve the quality of the framed image and maintain its dimensionality and aspect ratio, the portion outside the framed part is not removed but masked with a light green color.

Light green color is selected apart from other color such as black, white, brown, etc. for masking the non-eye region. It is because after careful analysis of the eye images in the training dataset light green color is found to be not present in the original images in the training dataset. If another color such as brown, black, or white color is used for framing, there will be a possibility that the model would misinterpret it for some feature/area present inside the eye region having that color.

In step S230, the processor 110 performs the coronavirus test (or referred to as coronavirus infection probability assessment) by inputting the processed eye image into a ML model to obtain an assessment result corresponding to the processed eye image, wherein the assessment result indicates that the processed eye image is classified as a first type or a second type. The ML model comprising of a CNN, a DCNN, a SVM, or a combination thereof.

Specifically, after receiving the processed eye image, the ML model processes it and then delivers an unambiguous, comprehensible result. The assessment result 310 may, in the form of simple graphics and text, indicate the eye image (either the initial image or the processed eye image) is classified as one of two types, e.g., positive 311 or negative 312. When the eye image is classified as the first type, this is because the ML model assigned the image as sufficiently similar to a plurality of eye images from patients having coronavirus disease, and therefore the user is positive for infection. Likewise, when the eye image is classified as the second type, the assessment result means the ML model assigned the image as similar to eye images from people who do not have coronavirus, so the user is negative for infection.

Next, in step S240, the processor 110 sends result data comprising the assessment result and the identification information to the mobile computing device D1 or an electronic device D2. The result data RD can be sent to the client application/webpage running on the mobile computing device D1 and inform the user U1 what the diagnostic result of his/her eye image is. The result data RD also can be sent to the client application/webpage running on the electronic device D2 (or electronic device D3), providing the coronavirus test result of user U1 for the related medical personnel U2's reference.

In an embodiment, to ensure scalability and availability of the system, the ML model is trained/tested and served through cloud (e.g., AWS). For training and testing the ML model, AWS Sagemaker is used. A WebApp has been deployed which is an ec2 instance in AWS which hosts the trained ML model and performs the assessment of the received eye image.

Ultimately, this invention provides a direct clinical benefit not only to providers and patients, but also to hospitals, health departments and other testing centers, by offering a rapid, inexpensive, noninvasive testing method to evaluate whether a person is infected or not with coronavirus.

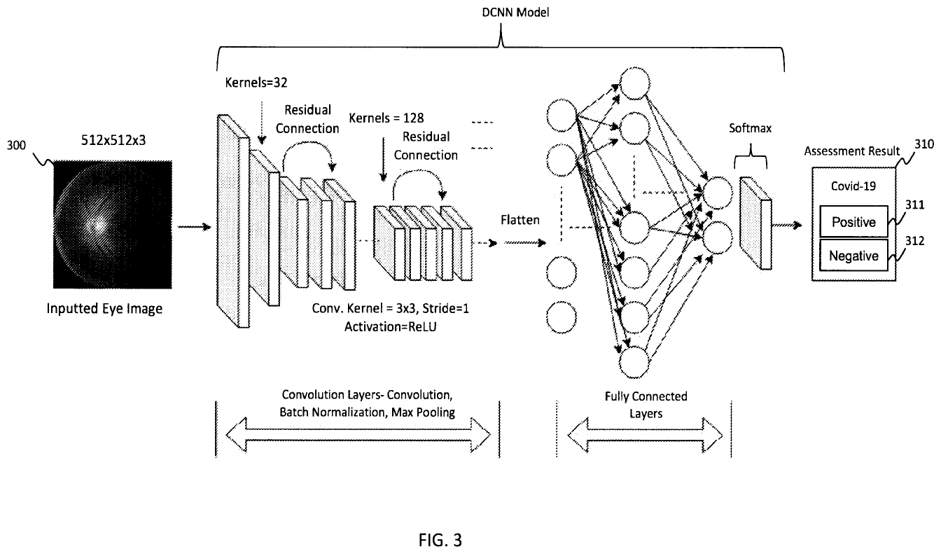

Referring to FIG. 3 for the following description. The constructed DCNN is a deep neural network comprising numerous layers for feature extraction and classification. It incorporates convolution and fully connected layers along with residual blocks involving skip connections.

In the embodiment, the DCNN's architecture uses a plurality of convolution layers, wherein a kernel size of a convolution operation performed on the convolution layers is 3×3 and a stride of the convolution operation is 1; a plurality of max-pooling layers; and a plurality of fully connected layers, wherein a flattening operation performed after the last max-pooling layer transfers data outputted from the last max-pooling layer to the first of the fully connected layers.

Deep neural networks typically suffer from the problem of vanishing gradient. However, introducing residual blocks with skip connections ensures that the gradient does not diminish as the data flows through the deeper layers. Batch normalization accelerates the training while L2 regularization is added to prevent over-fitting.

In the embodiment, multiple activation functions are used throughout the DCNN. These include rectified linear unit activation functions and softmax activation function. Specifically, one of the rectified linear unit activation functions is used after each of the convolution layers, wherein each one of the max-pooling layers is connected after each one of the rectified linear unit activation functions. The softmax activation function is engaged after the last of the fully connected layers, and it is configured to output the assessment result according to data output from the last of the fully connected layers.

The input processed eye image 300 of size 512×512 consisting of three channels (RGB) traverses through the DCNN in the following sequence. The convolution layers—stacked one after the other with varying numbers of kernels, comprising a convolution operation with a fixed kernel size of 3×3 with a stride of 1 unit, batch normalization and max pooling—ensure that the DCNN extracts sufficient features and transfers them to the subsequent layers. The skip connections ensure that the model is iteratively learning from the extracted features. This is followed by a flattening operation and a series of fully connected or dense layers. Finally, the data is passed to the layer with the softmax activation function that determines the conclusion (e.g., the assessment result 310). The hyper-parameters of the DCNN are fine-tuned iteratively to reach maximum validation accuracy over the validation dataset.

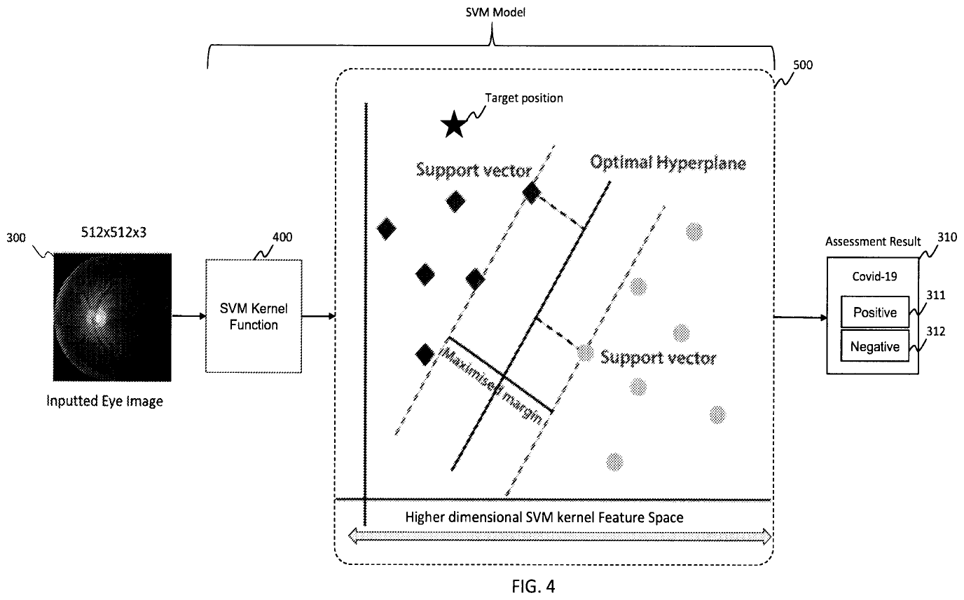

Referring to FIG. 4 for the following description. An architecture of the SVM includes a classifier 500 and one or more kernel functions 400. The classifier 500 having a higher dimensional SVM kernel feature space, wherein an optimal hyperplane is set to divide the higher dimensional SVM kernel feature space to two sub-spaces, wherein a first sub-space corresponds to the first type and a second sub-space corresponds the second type. The types of the kernel functions include: Linear Kernel, Radial Basis Function (RBF) Kernel, Polynomial Kernel and Sigmoid Kernel.

When the processed eye image 300 is inputted to the SVM, the kernel functions 400 transform the processed eye image 300 to a target feature vector, and the classifier 500 identifies a target position corresponding to the processed eye image 300 in the higher dimensional SVM kernel feature space according to the target feature vector. Then, the classifier 500 determines whether the target position belongs to the first sub-space or the second sub-space, so as to classify the processed eye image as the first type or the second type.

Specifically, the goal of the SVM is to create the best line or decision boundary that can segregate n-dimensional space into classes, such that the target position corresponding to the processed eye image 300 can be put in the correct sub-space. This best decision boundary is called the optimal hyperplane. During the training process, the SVM chooses the extreme points/vectors that help in creating the optimal hyperplane. These extreme cases are called support vectors. There could be many such hyperplanes that can be drawn to classify the data. However, the maximum margin hyperplane or optimal hyperplane is the one that has the maximum margin, wherein margin is the distance between two support vectors respectively belonging to two sub-spaces/classes.

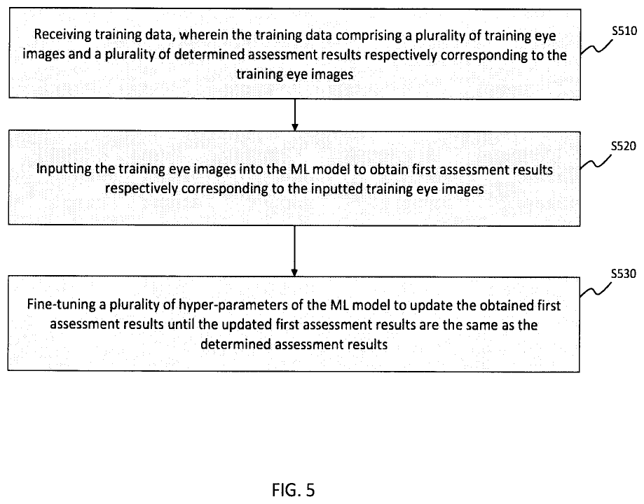

FIG. 5 details how the ML model achieves this optimal accuracy. Referring to FIG. 5 for the following description. In step S510, the processor 110 receives, via the communication circuit unit 130, training data TD, comprising a wide variety of training eye images and their respective determined results. For example, an eye image captured from the eye of a confirmed coronavirus patient serves as the training image, and it is integrated with the corresponding test result of “Positive” to generate one training datum. Conversely, an eye image captured from the eye of a person confirmed as free of coronavirus infection would serve as another training image, and when integrated with the corresponding test result of “Negative” this generates another training datum.

In step S520, the processor 110 inputs the training eye images into the ML model to generate first assessment results that respectively correspond to the training eye images.

Finally, in step S530, the processor 110 fine-tunes the hyper-parameters of the ML model to update its obtained first assessment results until they match the determined assessment results. Once this is accomplished, the processor 110 will conclude that the ML model is well-trained. In a further embodiment, the processor 110 may further input another batch of new training data TD to test or re-train the trained ML model via the above steps.

Regarding the SVM, the key parameters in SVM are: Gamma, which defines how far the influence of single training examples reaches with low values meaning ‘far’ and high values meaning ‘close’; C, which controls the cost of miscalculations. A small C makes the cost of misclassification low, and a large C makes the cost of misclassification high; and Kernel, which is a mathematical function used by the SVM. In an embodiment, the SVM can be trained by a standard function (“SVC( )” function) without doing hyper-parameter tuning and seeing its classification and confusion matrix. Those key parameters are then tuned to get the best results from the SVM. For example, a function of GridSearchCV is used for the training. The main idea is to create a grid of hyper-parameters and try all of their combinations. GridSearchCV takes a dictionary that describes the parameters that could be tried on the SVM model to train it. The grid of parameters is defined as a dictionary, where the keys are the parameters and the values are the settings to be tested, so that the best parameters for the SVM would be obtained. The goal in SVM is to find the hyperplane that maximizes the margin between the two classes.

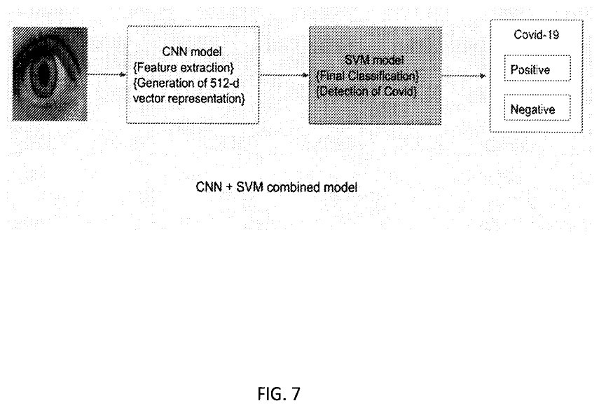

Referring to FIG. 7 for the following description. In one embodiment, the ML model is a combined CNN and SVM model, which comprises a CNN model for feature extraction and an SVM model for classification. In one embodiment, ImageNet is used for the CNN model with its architecture updated by removing the last couple of layers. A flattening layer followed by some fully connected layers are added to the architecture so that a 512-dimensional vector is generated from the model as its output. The 512-dimensional vector latent representation captures the important and intrinsic features of the input image. This 512-dimensional vector is fed as the input to the SVM model. The SVM model has an RBF kernel and is used for coronavirus infection detection.

During training, the combined CNN and SVM model is trained end to end. The training loss gets back propagated throughout the entire network architecture of the model. This ensures that the combined CNN and SVM model architecture is optimized for the purpose of coronavirus detection. The CNN module is optimized for performing the task of extracting the best latent image features and the SVM module is optimized for using the vector representation for efficient coronavirus detection.

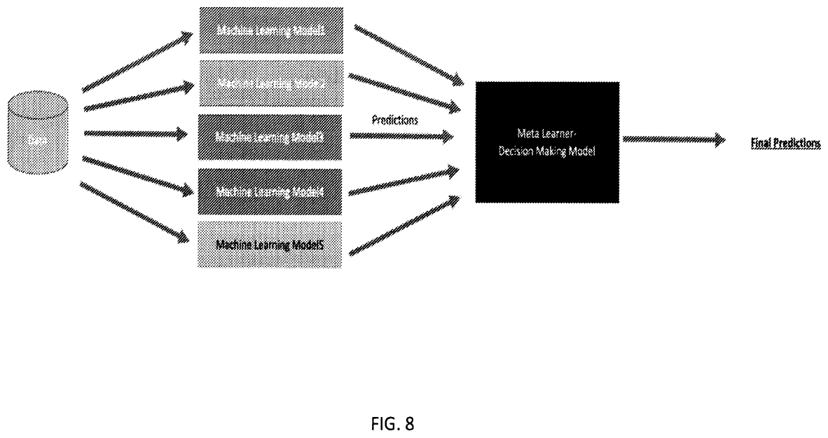

Referring to FIG. 8 for the following description. In another embodiment, provided is a Master-Meta model that uses multiple ML models to obtain coronavirus infection assessment results from the input processed eye images. In this Master-Meta model, the results from all of the ML models are taken for a voting. This reduces the overall incorrect predictions. Meta model is mathematically backed, and is widely used in the ML community. The concept of the Meta model in itself is not unique. The Master-Meta model adapts the Meta model as part of its building blocks. The uniqueness comes as that specific combination of models, which has not been used for the creation of the meta model.

In one embodiment, the Master-Meta is implemented by aggregating the output from each model with two objectives: reducing the model error and maintaining its generalization. When multiple ML models are generating prediction results, to combine the multiple models, an aggregating method is needed. Three main techniques can be used:

Max Voting: the final prediction in this technique is made based on majority voting from the different models for classification problems.

Averaging: typically used for regression problems where predictions are averaged; the probability can be used as well, for instance, in averaging the final classification.

Weighted Average: sometimes, weights are needed in some models/algorithms when producing the final predictions.

The reduction of model error using multiple ML models can be illustrated mathematically as below:

In one example with five models, suppose each model has an accuracy of 90%. If only one model is used for prediction, the probability of incorrect prediction is 0.1=10%. However, if a majority voting is used among the five models, then an incorrect prediction happens when:
1. All five models give incorrect prediction->Prob=0.1*0.1*0.1*0.1*0.1= 1/100000=0.00001;
2. Any four models give incorrect predictions->Prob=5C4*(0.1)*(0.1)*(0.1)*(0.1)*(0.9)=0.00045; and
3. Any three models give incorrect predictions->Prob=5C3*(0.1)*(0.1)*(0.1)*(0.9)*(0.9)=0.0081;
   The total probability of incorrect predictions is then=0.00001+0.00045+0.0081=0.00856=˜0.01=1%. Therefore, by this method, the probability of incorrect predictions is reduced by ten folds. Further, it is assumed that the predictions of all of the five models are mutually independent events.

The functional units of the apparatuses and the methods in accordance to embodiments disclosed herein may be implemented using computing devices, computer processors, or electronic circuitries including but not limited to application specific integrated circuits (ASIC), field programmable gate arrays (FPGA), and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments include computer storage media having computer instructions or software codes stored therein which can be used to program computers or microprocessors to perform any of the processes of the present invention. The storage media can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units in accordance to various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A computer-implemented method for performing a coronavirus testing using a machine learning (ML) model, the method comprising:
receiving examining data from a mobile computing device, wherein the examining data comprises an identification information related to the mobile computing device and an initial eye image captured on a user via an optical sensor of the mobile computing device;
performing a pre-processing operation to the initial eye image to obtain a processed eye image;
performing the coronavirus testing by inputting the processed eye image to the ML model to obtain an assessment result corresponding to the processed eye image, wherein the assessment result indicates that the processed eye image is classified as a first type or a second type; and
sending result data comprising the assessment result and the identification information to the mobile computing device or an electronic device.

2. The method of claim 1, wherein the pre-processing operation comprises:
an image framing step comprising:
detecting an eye region in the initial eye image;
determining coordinates of the eye region; and
masking portion outside of the coordinates with a masking shade to generate the processed eye image.

3. The method of claim 2, wherein the pre-processing operation further comprises:
in response to determining that the initial eye image is out-of-focus, performing a super resolution operation to the initial eye image to obtain the processed eye image; and
in response to determining that the initial eye image is underexposed or overexposed, adjusting a contrast value and a brightness value of the initial eye image to obtain the processed eye image having the adjusted contrast value within a predefined range and the adjusted brightness value within a further predefined range,
wherein the obtained processed eye image includes 512×512 pixels consisting of three channels of RGB information.

4. The method of claim 2, wherein the masking shade is of light green color.

5. The method of claim 1, wherein the initial eye image comprises:
an initial retinal image captured by performing a fundus photography on the user via the optical sensor of the mobile computing device; or
an initial eye region image captured by the optical sensor of the mobile computing device;
wherein the eye region includes at least retina, fundus, sclera, pupil, iris, cornea, conjunctiva, and lens of the eye.

6. The method of claim 1,
wherein when the processed eye image is classified as the first type, the assessment result indicates that the initial eye image is classified to a group of a plurality of eye images belonging to patients having coronavirus disease, and a coronavirus infection probability of the user is positive; and
wherein when the processed eye image is classified as the second type, the assessment result indicates that the initial eye image is classified to a further group of a plurality of further eye images belonging to people not infected with coronavirus disease, and the coronavirus infection probability of the user is negative.

7. The method of claim 1, wherein the ML model is a Master-Meta model, which obtains and aggregates multiple-ML model coronavirus infection assessment results from the processed eye images for reduction of the model error.

8. The method of claim 1, wherein the ML model is a combined Deep Convolution Neural Network (DCNN) and Support Vector Machine (SVM) model, which comprises a DCNN model for feature extraction and an SVM model for classification.

9. An analysis system for performing a coronavirus testing using a Machine Learning (ML) model, the system comprising:
a mobile computing device, configured to capture an initial eye image from a user;
an electronic device; and
an analysis server, comprising:
a communication circuit unit, configured to establish a network connection to the smartphone and the electronic device;
a storage circuit unit, configured to store programs; and
a processor, wherein the processor is configured to access and execute the programs to implement a coronavirus infection probability assessment method using the ML model, and the coronavirus infection probability assessment method comprises:
receiving examining data from the mobile computing device, wherein the examining data comprises an identification information related to the mobile computing device and the initial eye image captured on the user via an optical sensor of the mobile computing device;
performing a pre-processing operation to the initial eye image to obtain a processed eye image;
performing the coronavirus testing by inputting the processed eye image to the ML model to obtain an assessment result corresponding to the processed eye image, wherein the assessment result indicates that the processed eye image is classified as a first type or a second type; and
sending result data comprising the assessment result and the identification information to the mobile computing device or the electronic device.

10. The system of claim 9, wherein the pre-processing operation comprises:
an image framing step comprising:
detecting an eye region in the initial eye image;
determining coordinates of the eye region; and
masking portion outside of the coordinates with a masking shade to generate the processed eye image.

11. The system of claim 10, wherein the pre-processing operation further comprises:
in response to determining that the initial eye image is out-of-focus, performing a super resolution operation to the initial eye image to obtain the processed eye image; and
in response to determining that the initial eye image is underexposed or overexposed, adjusting a contrast value and a brightness value of the initial eye image to obtain the processed eye image having the adjusted contrast value within a predefined range and the adjusted brightness value within a further predefined range,
wherein the obtained processed eye image includes 512×512 pixels consisting of three channels of RGB information.

12. The system of claim 10, wherein the masking shade is of light green color.

13. The system of claim 9, wherein the initial eye image comprises:
an initial retinal image captured by performing a fundus photography on the user via the optical sensor of the mobile computing device; or
an initial eye region image captured by the optical sensor of the mobile computing device;
wherein the eye region includes at least retina, fundus, sclera, pupil, iris, cornea, conjunctiva, and lens of the eye.

14. The system of claim 9,
wherein when the processed eye image is classified as the first type, the assessment result indicates that the initial eye image is classified to a group of a plurality of eye images belonging to patients having coronavirus disease, and a coronavirus infection probability of the user is positive; and
wherein when the processed eye image is classified as the second type, the assessment result indicates that the initial eye image is classified to a further group of a plurality of further eye images belonging to people not infected with coronavirus disease, and the coronavirus infection probability of the user is negative.

15. The system of claim 9, wherein the ML model is a Master-Meta model, which obtains and aggregates multiple-ML model coronavirus infection assessment results from the processed eye images for reduction of the model error.

16. The system of claim 9, wherein the ML model is a combined Deep Convolution Neural Network (DCNN) and Support Vector Machine (SVM) model, which comprises a DCNN model for feature extraction and an SVM model for classification.
